(19) United States
(12) Patent Application Publication
(10) Pub. No.: US 2014/0346366 A1
(43) Pub. Date: Nov. 27, 2014

(54) SYSTEM AND METHOD FOR MEASURING CHLORINE CONCENTRATION IN FLY ASH CEMENT CONCRET

(71) Applicant: KING FAHD UNIVERSITY OF PETROLEUM AND MINERALS, DHAHRAN (SA)

(72) Inventors: AKHTAR ABBAS NAQVI, DHAHRAN (SA); MOHAMMED MASLEHUDDIN, DHAHRAN (SA); OMAR SAEED BAGHABRA AL-AMOUDI, DHAHRAN (SA); ZAMEER KALAKADA, JUBAIL (SA); FARIS AHMAD AL-MATOUQ, DHAHRAN (SA)

(73) Assignee: KING FAHD UNIVERSITY OF PETROLEUM AND MINERALS, DHAHRAN (SA)

(21) Appl. No.: 13/899,514

(22) Filed: May 21, 2013

(51) Int. Cl. G01N 23/222; G01N 33/38

(52) U.S. Cl. CPC G01N 23/222; G01N 33/383; G01N 2223/0745
USPC 250/393

(57) ABSTRACT

The system for measuring chlorine concentration in fly ash cement concrete utilizes a portable neutron generator and a gamma-ray detector for performing prompt gamma neutron activation analysis of chlorine concentration in a fly ash cement concrete specimen. The system includes a portable neutron generator for generating a pulsed neutron beam having a neutron energy of approximately 2.5 MeV and a gamma-ray detector, such as a bismuth germanate (BGO) gamma-ray detector. A moderator having opposed first and second faces is further provided. The first face is positioned adjacent a target plane of the portable neutron generator, and the second face is adapted for positioning adjacent the fly ash cement concrete specimen. A detection axis of the gamma-ray detector is angled at approximately 45° with respect to an axis of symmetry of the fly ash cement concrete specimen.

### BACKGROUND OF THE INVENTION

1. Field of the Invention

The present invention relates to a system and method for measuring chlorine concentration in fly ash cement concrete.

2. Description of the Related Art

Corrosion of reinforcing steel in concrete is mainly attributed to the diffusion of chloride ions to the steel surface. Out of the numerous measures adopted to minimize reinforcement corrosion, one prominent method requires making the concrete dense and impermeable to chloride diffusion to the steel surface. Pozzolanic materials, such as fly ash (FA), silica fume (SF), blast furnace slag (BFS) and superpozz (SPZ), are added to concrete as a partial replacement of Portland cement to make it dense and impermeable. Fly ash is a fine, glass-like powder recovered from gases in a coal-fired electric power generation facility. It is a pozzolanic substance, containing aluminous and siliceous material that forms cement in the presence of moisture. When mixed with lime and water, it forms a compound similar to Portland cement. It should be noted that SPZ is a finely ground FA.

Due to the small pore and capillary structure of concrete and the ongoing pozzolanic reaction that makes the pores even smaller and the capillaries discontinuous, a reduced quantity of chlorides diffuse to the steel surface. The dense nature of FA cement concrete greatly reduces the amount of chlorides diffusing through the concrete structure, and therefore the extent of reinforcement corrosion is decreased, extending the life span of the structure. Further, due to an increased amount of calcium silicate hydrate, which is a product of the pozzolanic reaction between fly ash and calcium hydroxide, more chlorides are chemically bound in the cement matrix, rendering them harmless.

Preventive measures against corrosion of reinforcing steel also require monitoring of chloride concentration in concrete, and non-destructive techniques are preferred over the conventional methods. The prompt gamma neutron activation (PGNAA) technique has been successfully applied to determine the elemental composition of bulk samples non-destructively. PGNAA is a very widely applicable technique for determining the presence and amount of many elements simultaneously in samples ranging in size from micrograms to many grams. It is a non-destructive method, and the chemical form and shape of the sample are relatively unimportant. Typical measurements take from a few minutes to several hours per sample.

In PGNAA, the sample is continuously irradiated with a beam of neutrons. The constituent elements of the sample absorb some of these neutrons and emit prompt gamma-rays, which are measured with a gamma-ray spectrometer. The energies of these gamma-rays identify the neutron-capturing elements, while the intensities of the peaks at these energies reveal their concentrations. The amount of analyte element is given by the ratio of count rate of the characteristic peak in the sample to the rate in a known mass of the appropriate elemental standard irradiated under the same conditions. Typically, the sample will not acquire significant long-lived radioactivity, and the sample may be removed from the facility and used for other purposes. One of the typical applications of PGAA is its use as an online belt elemental analyzer or bulk material analyzer used in cement, coal and mineral industries.

The chloride concentration in FA, SF and BFS blended cement concretes has been previously measured using prompt gamma-ray intensities transmitted through the concrete specimen in the accelerator-based PGNAA setup. Those studies were carried out to gather data about the applicability of PGNAA technique for developing the MDC data of chlorine in the chloride-contaminated FA, SF and BSF cement concretes. Although that setup was suitable for laboratory tests, it was not suitable for use in the field due to the large size, sensitivity and expense of the equipment involved.

Thus, a system and method for measuring chlorine concentration in the fly ash cement concrete solving the aforementioned problems, particularly size, sensitivity and expense, is desired.

### SUMMARY OF THE INVENTION

The present system for measuring chlorine concentration in fly ash cement concrete utilizes a portable neutron generator and a gamma-ray detector for performing prompt gamma analysis of chlorine concentration in a fly ash cement concrete specimen. The system includes a portable neutron generator for generating a pulsed neutron beam having neutron energy of approximately 2.5 MeV, and a gamma-ray detector, such as a bismuth germanate (BGO) gamma-ray detector.

A moderator having opposite first and second faces is further provided. The first face is positioned adjacent a target plane of the portable neutron generator, and the second face is adapted for positioning adjacent the fly ash cement concrete specimen. The detection axis of the gamma-ray detector is angled at approximately 45° with respect to an axis of symmetry of the fly ash cement concrete specimen. Preferably, gamma-ray shielding is positioned about the gamma-ray detector, and neutron shielding is positioned between the portable neutron generator and the gamma-ray detector.

These and other features of the present invention will become readily apparent upon further review of the following specification.

### BRIEF DESCRIPTION OF THE DRAWINGS

FIG. 1 is a diagram illustrating a system for measuring chlorine concentration in fly ash cement concrete according to the present invention.

FIG. 2 is a graph illustrating gamma-ray yield as a function of gamma-ray energy for four fly ash cement concrete specimens having 0.8, 1.5, 2.5, or 3.5 wt % (weight of cement) chlorine contamination, respectively, and a control specimen with no chloride contamination, the graph being produced using the system for measuring chlorine concentration in fly ash cement concrete.

FIG. 3 is a graph illustrating the subtracted spectra of chlorine in gamma-ray yield as a function of gamma-ray energy for four superpozz (SPZ) cement concrete specimens containing 0.8, 1.5, 2.5 or 3.5 wt % chlorine, respectively, the graph being produced using the system for measuring chlorine concentration in fly ash cement concrete.

FIG. 4 is a graph illustrating gamma-ray yield as a function of chlorine concentration in the fly ash cement concrete specimens of FIG. 2.

Similar reference characters denote corresponding features consistently throughout the attached drawings.

### DETAILED DESCRIPTION OF THE PREFERRED EMBODIMENTS

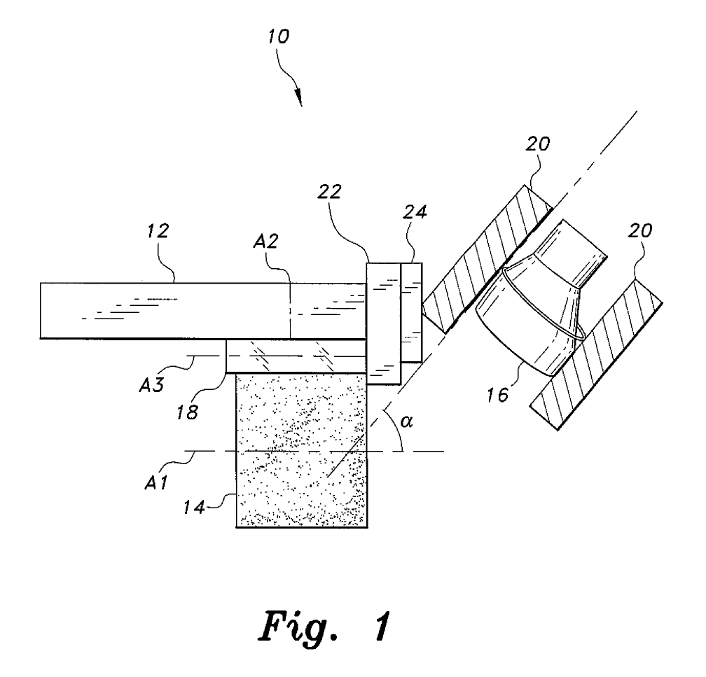

As shown in FIG. 1, the system 10 for measuring chlorine concentration in fly ash cement concrete uses a portable neutron generator 12 to generate neutrons for scattering within a fly ash cement concrete specimen 14 for PGNAA. The resultant gamma-rays caused by the scattering within the sample 14 are detected by a gamma-ray detector 16. The fly ash cement concrete specimen 14 is placed on one side of the neutron generator's target-plane location, so that the symmetry axis A1 of the specimen 14 is aligned at a right angle to the neutron generator's target-plane axis A2.

Preferably, a moderator 18, formed from high-density polyethylene (HDPE) or the like, is placed between the sample 14 and the neutron generator 12, so that the symmetry axis A3 of the moderator is aligned parallel with the symmetry axis A1 of the concrete specimen 14. The gamma-ray detector 16 preferably views the concrete specimen at an angle of 45° with respect to the symmetry axis A1 of sample 14. In FIG. 1, this is represented by angle α, which is equal to 45°.

It should be understood that any suitable type of portable neutron generator may be used for the PGNAA. One such portable neutron generator is the Thermo Scientific® MP 320 neutron generator, manufactured by Thermo Fisher Scientific, Inc. of Delaware. It should be further understood that any suitable type of moderator may be utilized. An exemplary moderator may be formed from HDPE and have a substantially cylindrical shape, for example. Exemplary dimensions for such a moderator include a diameter of approximately 25 cm and a height of approximately 8 cm. Such a moderator could be used with, for example, a substantially cylindrical sample 14. Exemplary dimensions used with the moderator described above could include a diameter of approximately 25 cm and a height of approximately 9 cm. Any suitable type of gamma-ray detector 16 may be used, such as a bismuth germanate (BGO) gamma-ray detector, for example. Typical BGO gamma-ray detectors are substantially cylindrical, having diameters of approximately 5 cm and heights of approximately 5 cm.

In order to prevent undesired gamma-rays and neutrons from reaching the detector, lead shielding 20, tungsten shielding 22 and paraffin neutron shielding 24 are inserted between the portable neutron generator 12, the moderator 18 and the gamma-ray detector 16, as shown in FIG. 1. Typically, such paraffin neutron shielding 24 is made of a mixture of paraffin and lithium carbonate mixed in equal weight proportions.

In our experiment, four fly ash cement concrete specimens were prepared with 0.8, 1.5, 2.5, or 3.5 wt % chloride contamination, respectively. The chlorine concentration in the chloride-contaminated fly ash cement concrete specimens was measured using the system 10 described above. The fly ash cement concrete specimens were prepared by mixing 20 wt % fly ash as a replacement of cement. The chemical compositions of Portland cement and fly ash are shown below in Table 1. All the concrete ingredients were thoroughly mixed in a revolving type drum mixer and thereafter poured in a specially designed cylindrical mold having a length of 14 cm and a radius of 12.5 cm. The concrete specimens were de-molded after one day, and then cured by covering them with wet burlap for 13 days and drying in an electric oven at a temperature of 70° C. for two days.

TABLE 1
Chemical Composition (wt %) of Portland Cement and Fly Ash

| | Type I | Fly |
|---|---|---|
| Compound | cement | ash |
| SiO₂ | 20.52 | 52.30 |
| Al₂O₃ | 5.64 | 25.20 |
| Fe₂O₃ | 3.80 | 4.6 |
| CaO | 64.35 | 10.0 |
| CaCO₃ | — | — |
| MgO | 2.11 | 2.20 |
| SO₃ | 2.1 | 0.60 |
| K₂O | 0.36 | 0.10 |
| Na₂O | 0.19 | 0.10 |

The chloride-contaminated fly ash cement concrete specimens 14 were then irradiated by the portable neutron generator 12. A pulsed beam of 2.5 MeV neutrons was produced via D(d,n) reaction using the Thermo Scientific® MP 320 portable neutron generator described above. The neutron generator was operated with a 70 keV deuteron beam with a pulse width of 5 ms and a frequency of 250 Hz. It was found that the pulsed neutron beam improved the signal-to-background-noise ratio in the PGNAA. The typical beam current of the generator was 70 μA. The thermal neutron spectra were acquired in PC-based data acquisition system utilizing multichannel buffer modules. The prompt gamma-ray data from the chloride-contaminated fly ash cement concrete specimens were acquired for 120 minutes. For background subtraction, prompt gamma-ray data were also acquired from a fly ash cement concrete specimen prepared without chloride contamination.

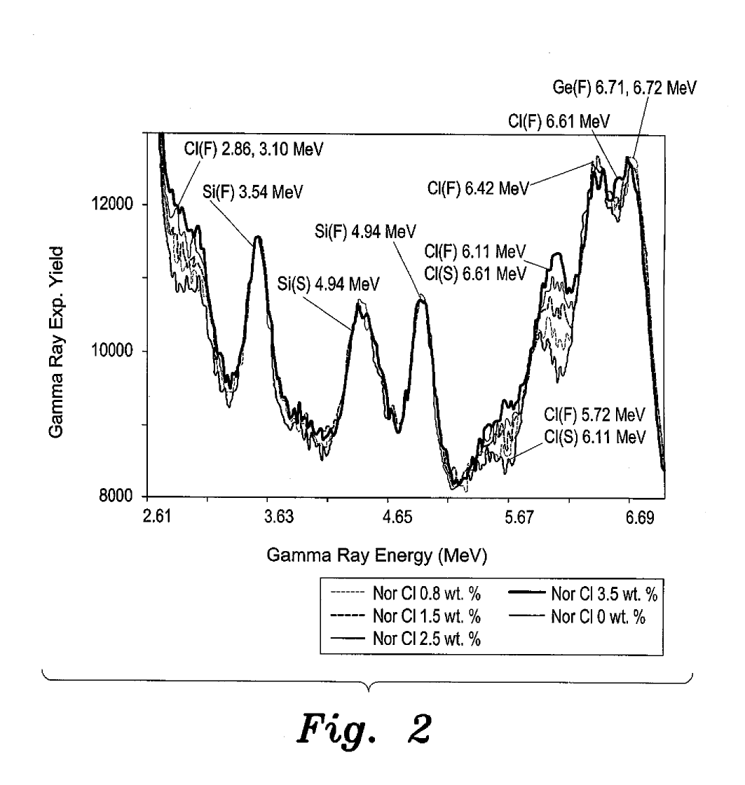

FIG. 2 shows the prompt gamma-ray spectra from fly ash cement concrete specimens containing 0.8, 1.5, 2.5, or 3.5 wt % chloride for 2.6 MeV gamma-ray energy. FIG. 2 shows chlorine prompt gamma-rays interfering with prompt gamma-rays from the materials in the fly ash cement concrete and the BOO gamma-ray detector. The full energy (F) and single escape (S) peaks of the prompt gamma-rays are shown in FIG. 2.

The partial elemental cross section in barns σ_{γ}^{z}(E_{γ}) for the production of gamma-rays E_{γ} from various elements Z in concrete (assuming natural abundance) are shown in Table 2, and the prompt gamma-ray partial elemental cross sections in barns σ_{γ}^{z}(E_{γ}) for chlorine are listed below in Table 3.

TABLE 2
Energies and partial elemental cross section σ_{γ}^{z}(E_{γ})-barns
of capture gamma-rays of concrete

| | Gamma-ray energy | |
|---|---|---|
| Element | (MeV) | σ_{γ}^{z}(E_{γ})-barns |
| Calcium | 1.942 | 0.352 |
| | 4.418 | 0.0708 |
| | 6.420 | 0.176 |
| Silicon | 3.539 | 0.1190 |
| | 4.934 | 0.1120 |
| Aluminum | 1.779 | 0.232 |
| | 7.724 | 0.0493 |
| Iron | 7.631 | 0.653 |
| | 7.646 | 0.549 |
| Hydrogen | 2.223 | 0.3326 |

TABLE 3
Energies and partial elemental cross section σ_{γ}^{z}(E_{γ})-barns of prominent
capture gamma-ray of BGO detector material and chlorine

| | Gamma-ray energy | |
|---|---|---|
| Element | (keV) | σ_{γ}^{z}(E_{γ})-barns |
| Bi | 162 | 0.008 |
| | 320 | 0.0115 |
| | 674 | 0.0026 |
| | 2505 | 0.0021 |
| | 2828 | 0.00179 |
| | 4054 | 0.0137 |
| | 4171 | 0.0171 |
| Cl | 2863 | 1.820 |
| | 3062 | 1.130 |
| | 5715 | 1.82 |
| | 6111 | 6.59 |
| | 6620 | 2.530 |
| | 6628 | 1.470 |
| Ge | 175 | 0.164 |
| | 493 | 0.133 |
| | 500 | 0.162 |
| | 596 | 1.100 |
| | 608 | 0.250 |
| | 868 | 0.553 |
| | 961 | 0.129 |
| | 1101 | 0.134 |
| | 1204 | 0.141 |
| | 1472 | 0.083 |
| | 5450 | 0.028 |
| | 5518 | 0.029 |
| | 5817 | 0.028 |
| | 6037 | 0.045 |
| | 6117 | 0.043 |
| | 6251 | 0.019 |
| | 6276 | 0.021 |
| | 6390 | 0.030 |
| | 6418 | 0.018 |
| | 6707 | 0.039 |
| | 6717 | 0.020 |
| | 6916 | 0.031 |
| | 7091 | 0.017 |
| | 7260 | 0.027 |
| | 7415 | 0.016 |
| | 8030 | 0.012 |
| | 8498 | 0.012 |
| | 8731 | 0.013 |

The calcium and silicon prompt gamma-ray peaks are located on the left-hand side of the BGO detector sum peak in FIG. 2. The full energy peak of calcium Ca(F) at 6.42 MeV can be seen to be interfering with the full energy peaks of Ge(F) at 6.71 and 6.72 MeV of BGO detector material. FIG. 2 also shows full energy prompt gamma-ray peaks from silicon Si(F) at 4.94 MeV and 3.54 MeV, and a peak at 4.44 MeV, which includes the single escape events from 4.94 MeV peak.

Several prompt gamma-rays are seen to be emitted by chlorine due to capture of thermal neutrons. In our experiment, only chlorine prompt gamma-rays with energies in excess of 2.66 MeV were considered. Due to the poor energy resolution of the BGO detector, chlorine prompt gamma-ray with energies of 2.86, 3.10, 5.72, 6.11 and 6.62 MeV could be resolved. The main feature of data in FIG. 2 is the increased intensities of some peaks due to the interference of chlorine gamma-rays with those of concrete and BGO material. The full energy peaks of 6.61 MeV gamma-rays from chlorine, and 6.42 MeV gamma-rays and 6.71-6.72 MeV gamma-rays from Ge in the BGO detector have strong interference. Although the full energy peak of 6.11 MeV prompt gamma-ray from chlorine interferes with the unlabeled single escape peak of 6.42 MeV gamma-ray from calcium, due to its highest intensity (6.59, 6.11 MeV), the prompt gamma-ray from chlorine is quite prominent in FIG. 2. Similarly, the single escape peak of 6.11 MeV chlorine interferes with the full energy peak of chlorine at 5.72 MeV.

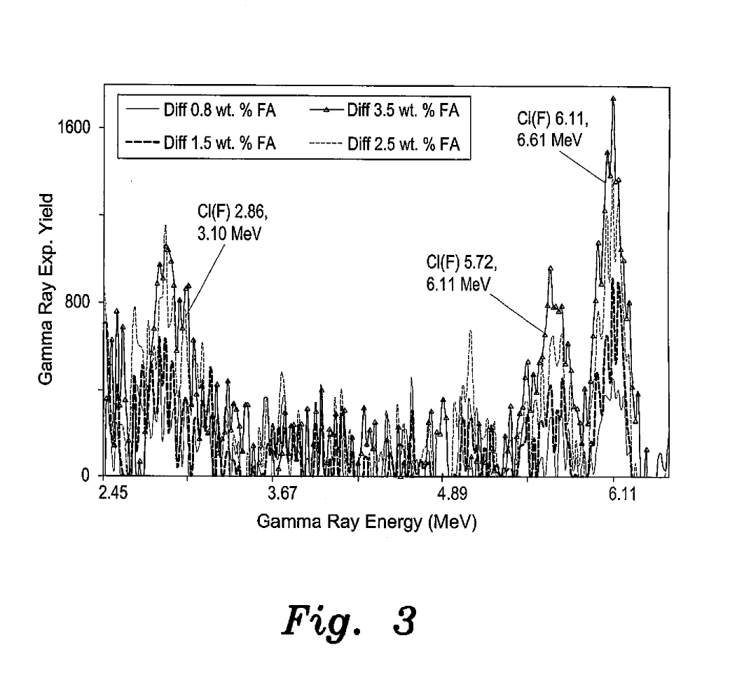

An unresolved broad prompt gamma-ray peak has been observed due to the interference of chlorine full energy peaks at 2.86 and 3.10 MeV. Finally, the chlorine gamma-ray yield from each of the chloride-contaminated fly ash cement concrete specimens was obtained after subtraction of normalized prompt gamma-ray spectra of an uncontaminated FA cement concrete specimen. FIG. 3 shows the subtracted spectra of chlorine prompt gamma-ray from superpozz (SPZ) cement concrete specimens containing 0.8, 1.5, 2.5, or 3.5 wt % chloride. Prominent chlorine full energy gamma-rays peaks corresponding to 2.86-3.10, 5.72 and 6.11 MeV energies are clearly seen in FIG. 3. The counts under each peak were integrated from the four spectra of fly ash cement concrete containing different chloride concentrations.

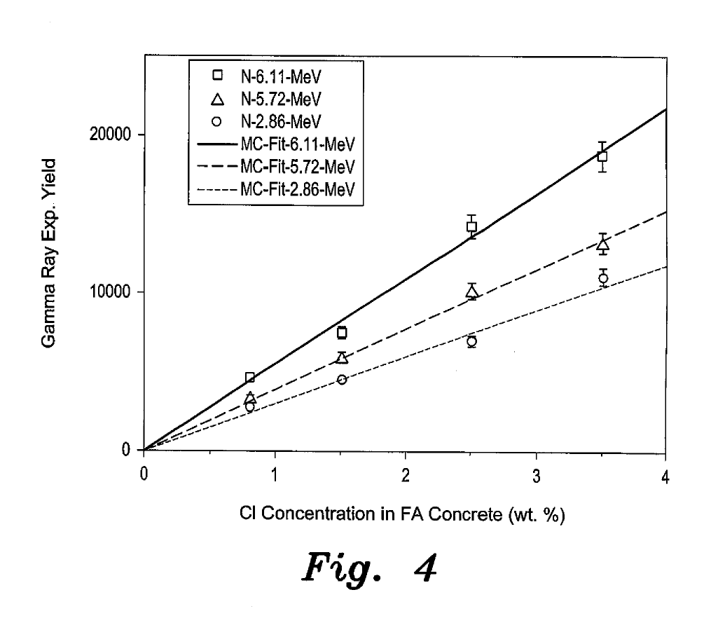

FIG. 4 shows the normalized integrated experimental yield of 2.86-3.10, 5.72 and 6.11 MeV chlorine gamma-rays as a function of chlorine concentration in the fly ash cement concrete specimen. Within the experimental uncertainties, there is an excellent agreement with the normalized calculated yield of the prompt gamma-rays from chlorine in fly ash cement concrete (shown as the solid line in FIG. 4), obtained through Monte Carlo calculations using MCNP4C code.

The minimum detectable concentration (MDC) and associated error σ_{MDC} of chlorine in fly ash cement concrete was calculated from the integrated yield of prompt gamma-ray and corresponding chlorine concentration data as:

$$MDC = 4.653 ( \frac{C}{{N}_{P}} ) \sqrt{{N}_{B}}$$

$$and$$

$${σ}_{MDC} = ( \frac{C}{{N}_{P}} ) \sqrt{2 {N}_{B}}$$

for an elemental concentration C measured under a peak with net counts N_{P} and associated background counts N_{B} under the peak. Table 4, below, shows the MDC of chlorine in fly ash (FA) cement concrete specimens determined by system 10 for 6.11 and 2.86-3.10, 5.72 and 6.11 MeV chlorine prompt gamma-rays. Also included in Table 4 are the MDC of chlorine prompt gamma-rays in plain, fly ash, silica fume (SF) and blast furnace slag (BFS) cement concretes.

TABLE 4
Comparison of MDC of Chloride in Concrete using Transmission
and Reflection PGNAA Techniques

| Reflectance Mode | | | | | |
|---|---|---|---|---|---|
| Gamma- | | | Transmission Mode | | |
| ray | | Portland | | SF | |
| energy | FA cement | cement | FA cement | cement | BFS cement |
| (MeV) | concrete | concrete | concrete | concrete | concrete |
| 2.86-3.12 | 0.033 ± 0.010 | — | — | — | — |
| 5.72 | 0.031 ± 0.010 | 0.255 ± 0.050 | — | — | — |
| 6.11 | 0.032 ± 0.010 | 0.140 ± 0.068 | 0.038 ± 0.017 | 0.026 ± 0.008 | 0.035 ± 0.011 |

The MDC of chlorine prompt gamma-rays in the fly ash (FA) cement concrete specimens for system 10 have been measured as 0.032±0.012, 0.037±0.012, 0.035±0.012 wt % for 2.86-3.10, 5.72 and 6.11 MeV gamma-rays, respectively. The best value of MDC limit of chlorine in the FA cement concrete was found to be 0.032±0.012 for 2.86-3.10 MeV prompt gamma-rays. In spite of a reduction in the gamma-ray intensity (due to the backward angle of the gamma-ray detector and the relative smaller neutron flux from a portable neutron), the values of MDC measured in the experiment were comparable with the MDC value for 6.11 MeV chlorine prompt gamma-rays measured in FA, SF and BFS cement concrete specimens, measured using transmission-type PGNAA.

The maximum permissible limit of chloride concentration in Portland cement concrete according to the American Concrete Institute Committee 318 is 0.03 wt % (weight of concrete). Within the statistical uncertainty, the lower bound of MDC of chlorine measured in the present experiment meets the maximum permissible limit of 0.03 wt % of chloride set by the American Concrete Institute (ACI) Committee 318. Based on the data shown above, it can be seen that the portable neutron generator based PGNAA setup of system 10 can be used successfully for non-destructive analysis of chloride in the FA cement concrete.

It is to be understood that the present invention is not limited to the embodiments described above, but encompasses any and all embodiments within the scope of the following claims.

We claim:

1. A system for measuring chlorine concentration in fly ash cement concrete, comprising:
a portable neutron generator for generating a neutron beam;
a moderator having opposed first and second faces, the first face being positioned adjacent a target plane of the portable neutron generator, the second face being adapted for positioning adjacent a fly ash cement concrete specimen; and
a gamma-ray detector positioned for prompt gamma neutron activation analysis of chlorine concentration in the fly ash cement concrete specimen when the neutron beam impinges thereon.

2. The system for measuring chlorine concentration in fly ash cement concrete as recited in claim 1, further comprising gamma-ray shielding positioned about said gamma-ray detector.

3. The system for measuring chlorine concentration in fly ash cement concrete as recited in claim 2, further comprising neutron shielding positioned between said portable neutron generator and said gamma-ray detector.

4. The system for measuring chlorine concentration in fly ash cement concrete as recited in claim 1, wherein said gamma-ray detector has a detection axis at an angle of about 45° with respect to an axis of symmetry of the fly ash cement concrete specimen.

5. The system for measuring chlorine concentration in fly ash cement concrete as recited in claim 1, wherein said gamma-ray detector comprises a bismuth germinate gamma-ray detector.

6. The system for measuring chlorine concentration in fly ash cement concrete as recited in claim 1, wherein said moderator is substantially cylindrical.

7. The system for measuring chlorine concentration in fly ash cement concrete as recited in claim 6, wherein said moderator has a thickness of about 8 cm and a diameter of about 25 cm.

8. The system for measuring chlorine concentration in fly ash cement concrete as recited in claim 7, wherein said moderator is formed from high-density polyethylene.

9. The system for measuring chlorine concentration in fly ash cement concrete as recited in claim 1, wherein said neutron beam is a pulsed neutron beam having a neutron energy of about 2.5 MeV.

10. A method of measuring chlorine concentration in fly ash cement concrete, comprising the steps of:
generating a neutron beam with a portable neutron generator;
impinging the neutron beam on a fly ash cement concrete specimen; and
performing prompt gamma neutron activation analysis on gamma-rays emitted from the fly ash cement concrete specimen to measure chlorine concentration therein.

11. The method of measuring chlorine concentration in fly ash cement concrete as recited in claim 10, further comprising the step of passing the neutron beam through a moderator prior to the step of impinging the neutron beam on the fly ash cement concrete specimen.

12. The method of measuring chlorine concentration in fly ash cement concrete as recited in claim 11, wherein the step of generating the neutron beam comprises generating a pulsed neutron beam having a neutron energy of about 2.5 MeV.

13. A system for measuring chlorine concentration in fly ash cement concrete, comprising:
a portable neutron generator for generating a pulsed neutron beam having a neutron energy of about 2.5 MeV;
a moderator having opposed first and second faces, the first face being positioned adjacent a target plane of the portable neutron generator, the second face being adapted for positioning adjacent a fly ash cement concrete specimen; and
a gamma-ray detector positioned for prompt gamma neutron activation analysis of chlorine concentration in the fly ash cement concrete specimen when the neutron beam impinges thereon.

14. The system for measuring chlorine concentration in fly ash cement concrete as recited in claim 13, further comprising gamma-ray shielding positioned about said gamma-ray detector.

15. The system for measuring chlorine concentration in fly ash cement concrete as recited in claim 14, further comprising neutron shielding positioned between said portable neutron generator and said gamma-ray detector.

16. The system for measuring chlorine concentration in fly ash cement concrete as recited in claim 15, wherein said gamma-ray detector has a detection axis at an angle of about 45° with respect to an axis of symmetry of the fly ash cement concrete specimen.

17. The system for measuring chlorine concentration in fly ash cement concrete as recited in claim 16, wherein said gamma-ray detector comprises a bismuth germinate gamma-ray detector.

18. The system for measuring chlorine concentration in fly ash cement concrete as recited in claim 17, wherein said moderator is substantially cylindrical.

19. The system for measuring chlorine concentration in fly ash cement concrete as recited in claim 18, wherein said moderator has a thickness of about 8 cm and a diameter of about 25 cm.

20. The system for measuring chlorine concentration in fly ash cement concrete as recited in claim 19, wherein said moderator is formed from high-density polyethylene.
